SEQUENCE LISTING (11 sequences)

SEQ ID NO: 1 - DNA, 1532 bp, Zea mays
  CDS (2)..(208)
  Intron (209)..(322)
  CDS (323)..(781)
  Intron (782)..(880)
  CDS (881)..(1285)
gacgaagcggcactgcttgcgcttcaccaccgtcctggcgacgacgcttctctccgccaccgccgcctgcctcgacgtcggcttctacgacaggacatgccccactgccgagaccatcgtgcagcagaccgtggcggccgcgttcaggaacaactccggcgtcgctccggcgctgatccgcatgcacttccatgactgctttgtcagggtaattaagctcacgcatgcatgtggctagaagacatgctgtttttttttcctctccacacgagcgagtgactgtacgtacgcgcgcgggctctaaactccttgcttgctgcagggctgcgatggctcggtgctgatcgacacggtaggcaacctgacggcggagaaggacgcgccacccaacaaccccagcctccggttcttcgacgtggtcgaccgtgccaaggcgtcactggaggctcagtgccccggcgtggtctcctgcgccgacgtgctcgccttcgcggccagggacagcgtcgtgctctccggtggcctcggctaccaggtgccggccggacgccgtgacgggcggatatccaatgacaccgaagccctcaacaacctgcctccgccgttcttcaacgccaccgagctggcagacaggttcgcctccaagaacctcagtatcgaggacctggtcgtgctctccggcgcgcacaccatcggcgtctcgcactgcagcggcttcgccggcccgacagacctgaacggccccgttgaccggctctacaacttcagctcgcctgacggggtaggggcatcgtctgtcacctcgctctctgcaaaaccttgaatgcgaaaaaaagatgacccccggttattacttaccatgcattgctgtggtgtacagattgacccgacgctgagcaaagcctacgcatttcttctcaagagcatctgcccggccaacaccagccagttcttcccgaacacgacggtgttcatggacctcatcacgccggaaaggtttgacaacaagtactacgtcggcctgaccaacaacctgggcctcttcaagtcagacgtggcgctgctgaccaacgcgacgatgaaggccctggtcgactccttcgtgcgcagcgaggcgactttcaggaccaagtttgccaggtccatgatcaagatggggcagatcgaggtgctgacggggacgcagggcgagatcaggcgcaactgcagggtcatcaaccccgttagtgccaccgatgatgtcgtcctcgcccgcccatcaggattcactggagtggctgcgagctagctaaccaactgtcggttcatgcacatgcattgtgcagtgtgtgatgtctagtgtgagttgatgtgtgaaattgtaataagtaatgagcagattatcttggtaagctcgcttgttactgtggcaaaagttgttttgttgcatgacaaaaatgtatactcatcggattattgaaaacaaaactgatatttgtgttcaggcaaataatagttctacaaattccttataaacatatatatttgtcgat

SEQ ID NO: 2 - protein, 357 aa, Zea mays
TKRHCLRFTTVLATTLLSATAACLDVGFYDRTCPTAETIVQQTVAAAFRNNSGVAPALIRMHFHDCFVRGCDGSVLIDTVGNLTAEKDAPPNNPSLRFFDVVDRAKASLEAQCPGVVSCADVLAFAARDSVVLSGGLGYQVPAGRRDGRISNDTEALNNLPPPFFNATELADRFASKNLSIEDLVVLSGAHTIGVSHCSGFAGPTDLNGPVDRLYNFSSPDGIDPTLSKAYAFLLKSICPANTSQFFPNTTVFMDLITPERFDNKYYVGLTNNLGLFKSDVALLTNATMKALVDSFVRSEATFRTKFARSMIKMGQIEVLTGTQGEIRRNCRVINPVSATDDVVLARPSGFTGVAAS

SEQ ID NO: 3 - DNA, 1744 bp, Zea mays
  CDS (2)..(208)
  CDS (334)..(534)
  misc_feature (458)..(779): Insertion MITE
gacgaagcggcactgcttgcgcttcaccaccgtcctggcgacgacgcttctctcggccactgccgcctgcctcgacgtcggcttctacgacaggacatgccccactgccgagaccatcgtgcggcagaccgtggcggccgcgttcaggaacaactccggcgtcgctccggcgctgatccgcatgcacttccatgactgccttgtcagggtaatcaagcacactgcacgcatgcacgcatgtagctagacgacatgctgttttttttttctctccacacgagcgagtgacgtaagtacgcgcgcgggctctaactctaaactacttgcttgcagggctgcgatggctcggtgctggtcgacacggtgggcaacctgacggcggagaaggacgcgccacccaacaaccccagcctccggttcttcgacgtggtcgaccgtgccaagacggcactggaggctcagggtgtgtttggtttggcttttggttttggcttttgccccctaaaagccaaaagccaaccaaagggctggatctaggaagcagctttttctaaaagtcgactttctcgtagtgcaaaactgaaagcacccctggacctgcttttagtggcttttgaatggaactgtgaaaacatatatcaaagaacttttaacgacttctagtggttttcaccaaacgatttttagctttttaacagcacacagcctacagcagctttttccacagctcacagcccacagcaactttttccacagccacagcccaaccaaacagaccctcagtgccccggcgttgtctcctgcgccgacgtgctcgccttcgcggccagggacagcgtcgtgctctccggtggcctcggctaccaggtgccggccggacgccgtgacgggcggatatccaatgacaccgaagccctcaacaacctgcctccgccgttcttcaacgccaccgagctggcagacaggttcgcctccaagaacctcactatcgaggacctggtcgtgctctccggcgcgcacaccatcggcgtctcgcactgcagcggcttcgccggcccgacagacctgaacggccccgttgaccggctctacaacttcagctcgcctgacggggtagggacatcgtctgtcacctcgctctctctgtccaaaaccttgaatgcgaaaaaaagatgacccccggttacttaccattgctgtggtgtacagattgacccgacgctgagcaaagcctacgcatttcttctcaggagcatctgcccggccaacaccagccagttcttcccgaacacgacggtgttcatggacctcatcacgccggaaaggtttgacaacaagtactacgtcggcctgaccaacaacctgggcctcttcaagtcagacgtggcgctgctgaccaacgcgacgatgaaggccctggtcgactccttcgtgcgcagcgaggcgactttcaggaccaagtttgccaggtccatgatcaagatggggcagatcgaggtgctgacggggacgcagggcgagatcaggcgcaactgcagggtcatcaaccccgttagtgccaccgatgatgtcgtcctcgcccgcccatcaggattcactggagtggctgcgagctagctaaccaactgtcggttcatgcacatgcgttgtgcacagtgtgtgatgtctagtgtgagttgatgtgtgaaattgtaataagtaatgagcagattatcttagtaagctcgcttgttactgtggcaaa

SEQ ID NO: 4 - protein, 136 aa, Zea mays
  misc_feature (458)..(779): Insertion MITE
TKRHCLRFTTVLATTLLSATAACLDVGFYDRTCPTAETIVRQTVAAAFRNNSGVAPALIRMHFHDCLVRGCDGSVLVDTVGNLTAEKDAPPNNPSLRFFDVVDRAKTALEAQGVFGLAFGFGFCPLKAKSQPKGWI

SEQ ID NO: 5 - DNA, 16 bp, Zea mays
caccggagtggctgcg

SEQ ID NO: 6 - DNA, 26 bp, Zea mays
atcgacaaatatatatgtttataagg

SEQ ID NO: 7 - DNA, 29 bp, Zea mays
gacgaagcggcactgcttgcgcttcacca

SEQ ID NO: 8 - DNA, 29 bp, Zea mays
tgccacagtaacaagcgagcttaccaaga

SEQ ID NO: 9 - DNA, 24 bp, Zea mays
ggcactggaggctcagggtgtgtt

SEQ ID NO: 10 - DNA, 20 bp, Zea mays
aggagacaacgccggggcac

SEQ ID NO: 11 - DNA, 30 bp, Zea mays
cgtcaggttgcctaccgtgtcgatcagcac

(19) United States
(12) Patent Application Publication
(10) Pub. No.: US 2006/0282912 A1
(43) Pub. Date: Dec. 14, 2006

(54) Production of plants with improved digestibility having an inactive peroxidase

(75) Inventors: Carine Guillet, Quebec (CA); Yves Barriere, Cloue (FR); Alain Murigneux, la Roche Blanche (FR); Jean-Pierre Martinant, Vertaizon (FR); Elise Redondo, Clermont-Ferrand (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: GENOPLANTE-VALOR, Evry (FR)

(21) Appl. No.: 10/548,481

(22) PCT Filed: Mar. 10, 2004
(86) PCT No.: PCT/FR04/00569

§ 371 Date: Jul. 26, 2006

(30) Foreign Application Priority Data
Mar. 10, 2003 (FR) 0302954

(51) Int. Cl. A01H 1/00; A01H 5/00; C12Q 1/68; C12N 15/82; C12N 5/04

(52) U.S. Cl. 800/278; 800/320.1; 435/6; 435/412; 435/468

(57) ABSTRACT

The invention relates to improving the digestibility of a plant by total or partial inhibition of the expression and/or of the activity of the Pox3/U19 peroxidase of said plant. The invention also relates to the selection of plants with improved digestibility, in which the expression and/or the activity of the Pox3/U19 peroxidase is partially or totally inhibited.

The present invention relates to improving the digestibility of fodder plants, and more particularly of corn.

The use of corn as fodder, in particular in the form of ensilage, is increasingly widespread. This is because fodder corn has many advantages: its open field yield is relatively high, and it can be easily harvested and stored. It constitutes a food intake rich in energy, the nutritional qualities of which are stable, which can be supplemented in terms of proteins by means of protein-yielding plants or by means of cakes of oil- and protein-yielding plants such as soybean, and makes it possible to obtain in particular an even and high-level milk production.

The improvement of fodder corn varieties initially related mainly to increasing yield, to hardiness and to resistance to torrential rain (BARRIERE et al., Fourrages, 107-119, 2000). However, it has been observed that, in parallel, the food value that was not taken into account in the selection criteria had decreased on average and showed great variability from one hybrid to the other, resulting in substantial differences in milk production. A selection effort was therefore undertaken in order to improve the food value of fodder corns, in particular after the development of equations for predicting the energy value and the use of a reference enzymatic solubility (ANDRIEU, Prod. Anim., 273-274, 1995).

An important component of the food value is digestibility. For example, various experiments carried out with dairy cows have shown that the use of more digestible varieties allows an increase in milk production and better weight gain by the cows, when the level of supplementation does not allow the animals to express their potential with the normal varieties. In addition, these more digestible varieties allow the animals to reach their potential with a lower level of supplementation, which makes it possible in particular to reduce production costs.

An important factor that limits the digestibility of fodder plants is related to the presence, in the plant cell walls, of phenolic compounds, in particular of lignins. Lignins establish various types of bonds with the other parietal constituents and form a tight mesh that impairs the accessibility of the digestive enzymes to the parietal carbohydrates, the main energy sources for herbivores. The portion of nondigested residues varies during the plant's development. The degree of lignification increases during maturation of the plant and causes a decrease in its digestibility. However, there exists a genetic variability in the intensity and in the quality of lignification between lines or hybrids, for a given level of maturity, that is associated with a variability in digestibility (MECHIN et al., J. Sci. Food Agric., 80, 574-580, 2000). This variability is also illustrated by the modifications in amount and quality of lignin and the improvement in digestibility observed in the “brown-midrib” mutants, in particular the bm3 mutant.

Consequently, one of the preferred pathways for improving the food value of fodder corn concerns the selection or the production, by genetic engineering, of plants in which the lignins are qualitatively or quantitatively modified.

Lignins are insoluble polymers of 3 alcohol monomers or monolignols, that derive from the phenylpropanoid pathway (NEISH, Constitution and Biosynthesis of Lignin, eds New York: Springer Verlag, 1-43, 1968): p-coumaryl alcohol (H subunits), coniferyl alcohol (G subunits) and sinapyl alcohol (S subunits). In corn, the respective proportions of the H/S/G units are in the region of 3/35/62 (MECHIN, INAPG thesis, 2000). Each of these precursors can form various bonds with the others and thus constitute lignin. Other bonds can also be established with other parietal compounds (polysaccharides and proteins) so as to form a complex three-dimensional network. The monolignols release, via oxidation, a radical that allows them to spontaneously combine. The formation of these radicals is thought to depend on peroxidases and on laccases or on other oxidases. A considerable number of these enzymes in combination with regulatory proteins is thought to be necessary in the assembly of the H, G and S subunits (BOUDET, Plant Physiol. Biochem., 38, 81-96, 2000).

Although the mechanisms involved in vivo in lignin biosynthesis have not been completely elucidated, it is generally considered that laccases could be involved in dimer and trimer formation, whereas peroxidases would make it possible to obtain a greater degree of polymerization based on the dimers and trimers (ROS BARCELO, International Review of Cytology, 176, 87-132, 1997).

Peroxidases belong to a multigene family and are very widely represented in the plant genome. For example, the genome of Arabidopsis thaliana is thought to contain more than 70 peroxidases (WELINDER et al., Eur. J. Biochem., 269(24), 6063-6081, 2002).

In addition, since peroxidases are involved in very varied metabolic pathways, it is very difficult to determine which are involved in lignification (BOUDET et al, Plant Physiol. Biochem., 38, 81-96, 2000).

QUIROGA et al. (Plant Physiol., 122, 1119-1127, 2000) and OSTERGAARD (Plant Mol. Biol., 44, 231-243, 2000) have identified peroxidases that are involved in lignin synthesis in the tomato (TPX1) and in Arabidopsis thaliana (ATP A2). MOROSHI and KAJITA (Journal of Plant Research, 517-523, 2001) have succeeded in under-regulating a peroxidase involved in lignin synthesis in a tree. In this case, the decrease in activity of this enzyme results in an increase in the content of S subunits and in noncondensed bonds, and also a decrease in the amount of lignins in the wall.

The inventors have mapped the gene of a peroxidase, that they have named peroxidase Pox3/U19, on chromosome 6 (bin 6.06), and have established that a colocalization exists between this gene and QTLs for digestibility and for wall lignin content. This gene corresponds to the Pox3 sequence listed in GenBank under accession number AJ401276.

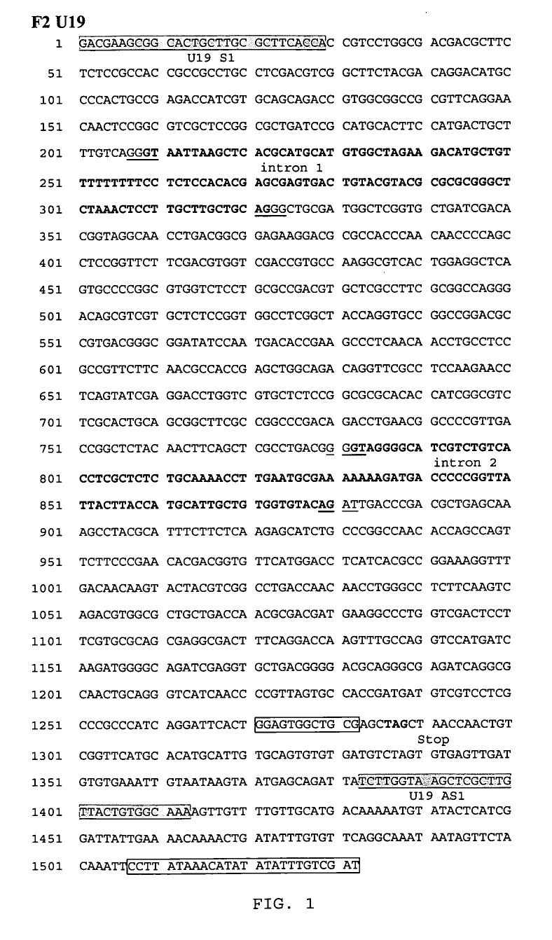

The Pox3/U19 genomic DNA sequence, obtained from the F2 corn line, is represented in FIG. 1, and also in the attached sequence listing under the number SEQ ID NO: 1. The corresponding polypeptide sequence is represented in the attached sequence listing under the number SEQ ID NO: 2.

The inventors have sequenced the genomic DNA of a length of approximately 1.7 kb of Pox3/U19 in 37 different corn lines or ecotypes and have thus been able to determine that the coding region consists of two introns, respectively of 127 and 111 base pairs, and of 3 exons. The polymorphism analysis carried out on these lines shows that there exists a polymorphic site every 57 base pairs on average, i.e. 31 SNPs (single nucleotide polymorphisms), over the entire sequence and 17 indels, for insertion-deletions, representing 20% of the total length of the sequence.

The inventors have also discovered that, in certain more digestible corn lines, the Pox3/U19 peroxidase gene is interrupted by a transposon fragment of MITE type (Miniature Inverted-repeat Transposable Element; WESSLER et al., Current Opinion in Genetics and Development, 5, 814-821, 1995), that introduces, at the beginning of the second exon, a stop codon which results in the production of a truncated and therefore nonfunctional protein. They have shown a significant correlation between the presence of this transposable element and the digestibility of the line.

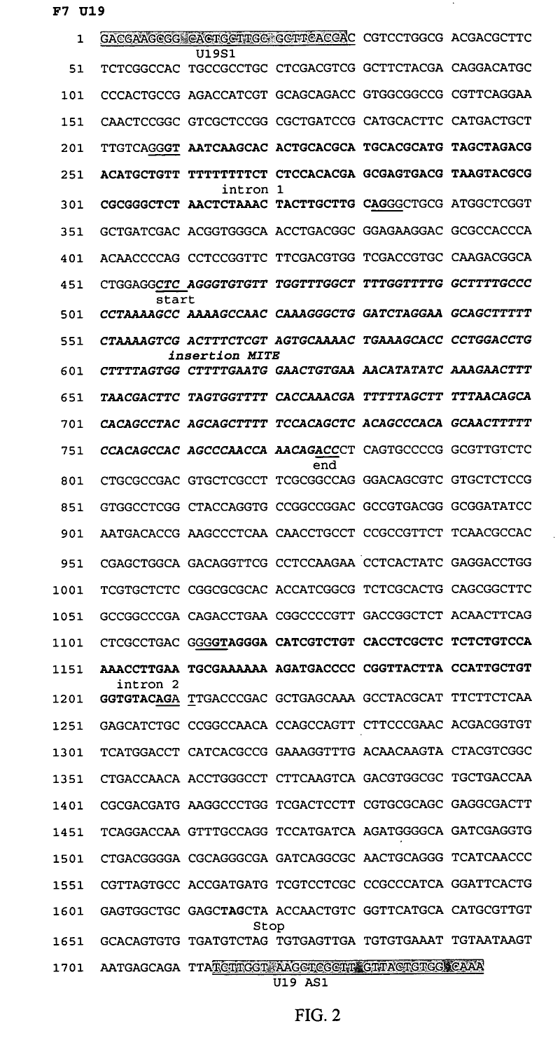

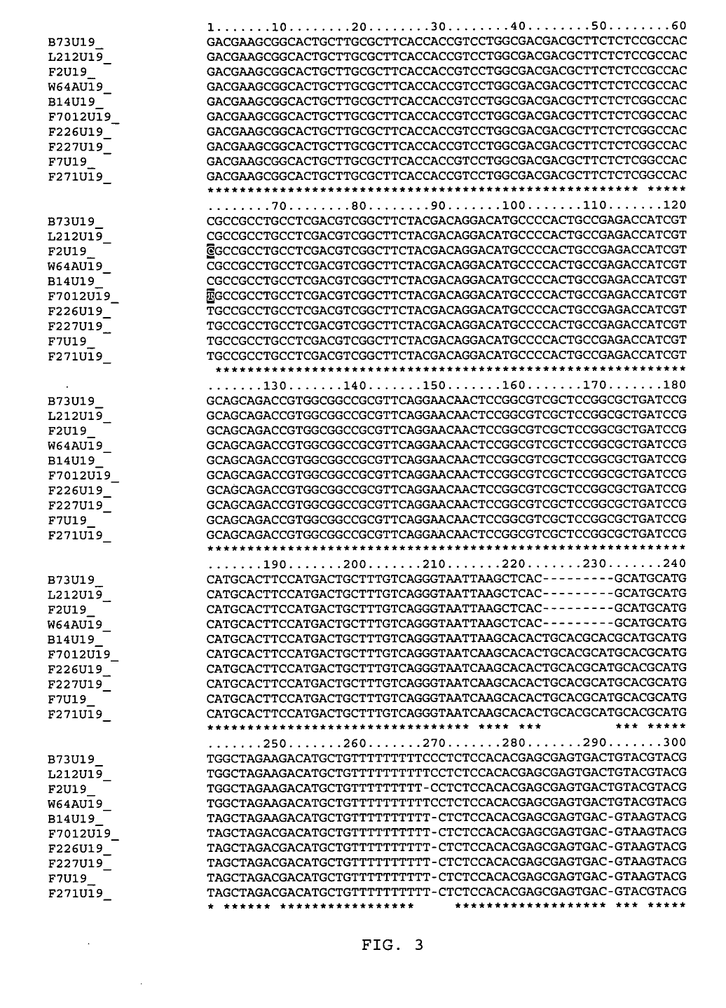

The Pox3/U19 genomic DNA sequence obtained from the high-digestibility F7 corn line is represented in FIG. 2, and also in the attached sequence listing under the number SEQ ID NO: 3. The corresponding polypeptide sequence is represented in the attached sequence listing under the number SEQ ID NO: 4.

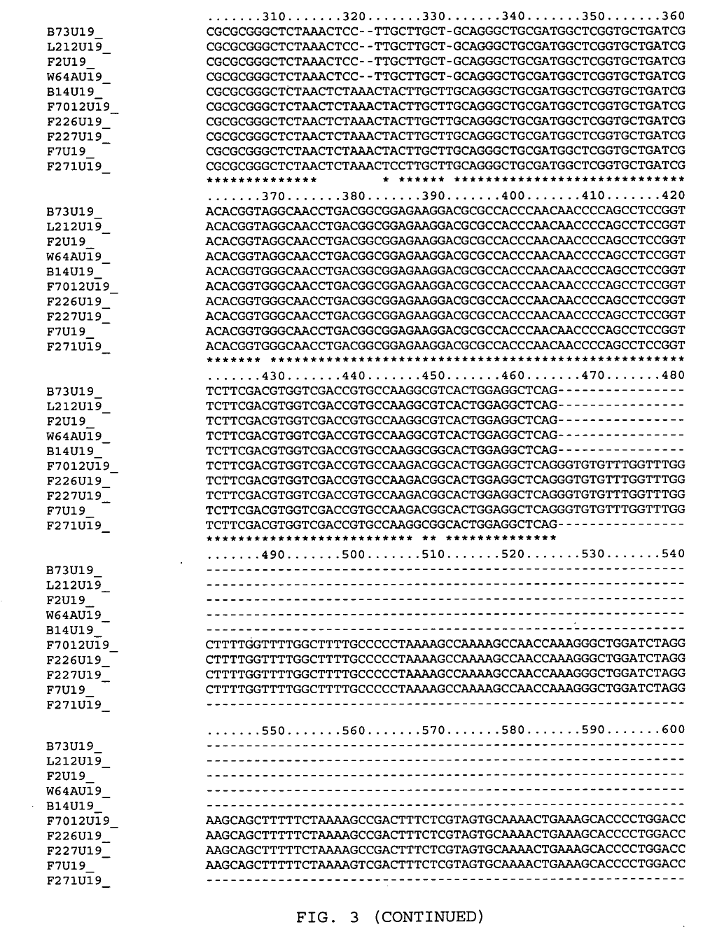

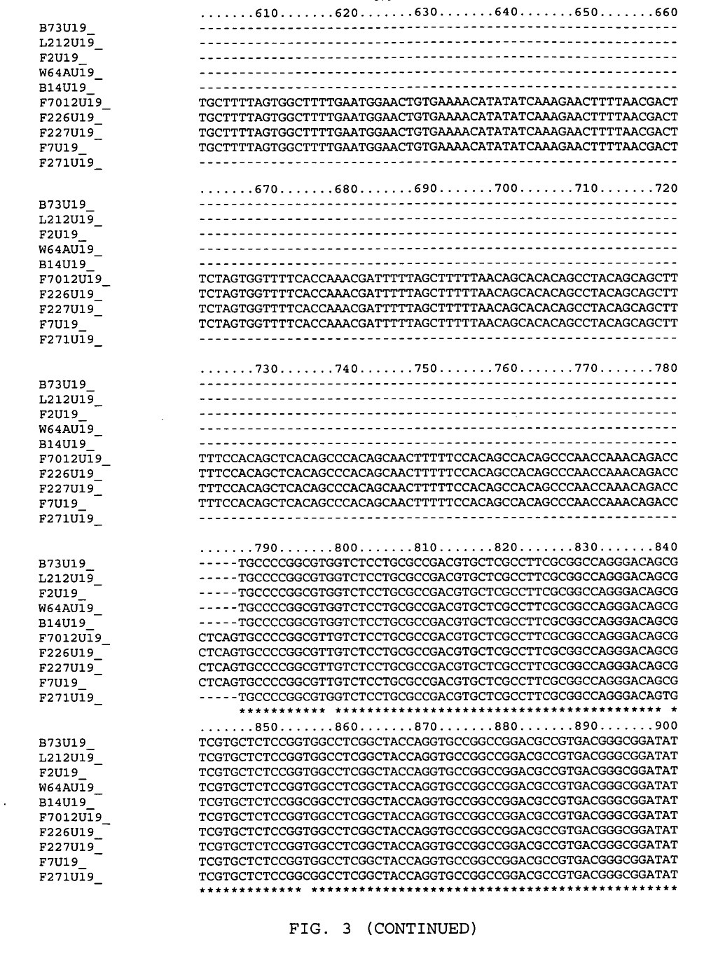

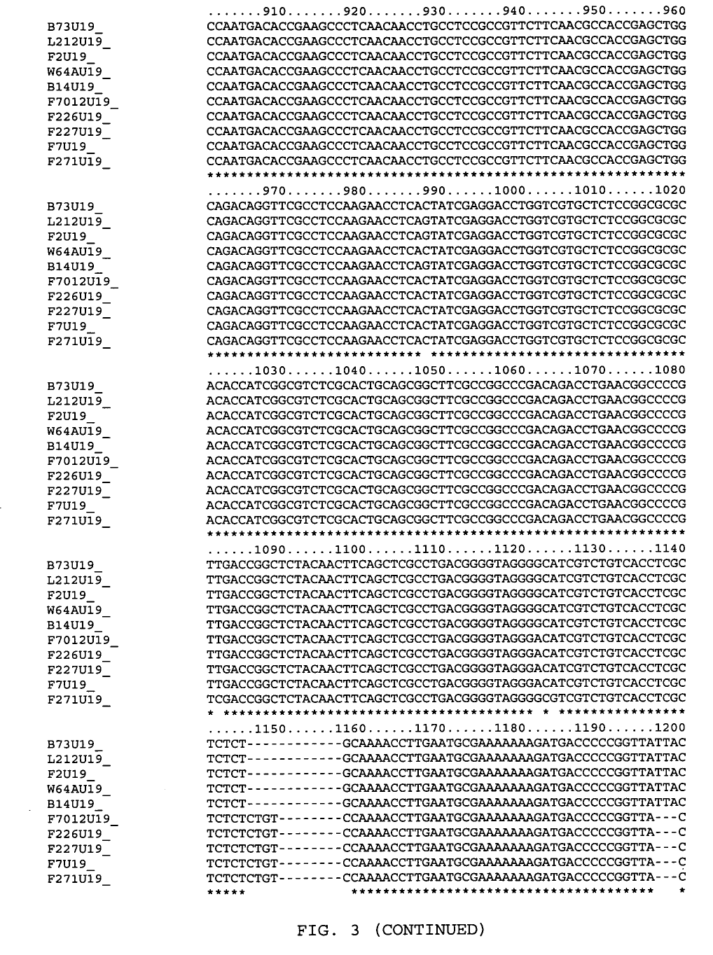

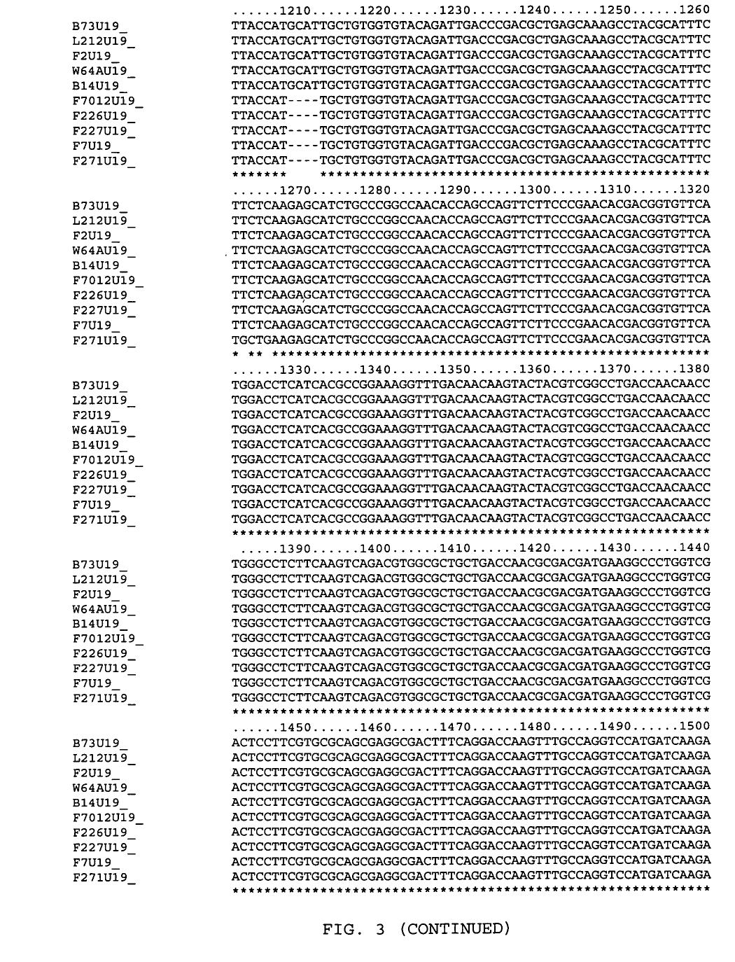

The alignment of Pox3/U19 genomic DNA sequences, obtained from the high-digestibility lines F226, F227, F7012 and F7, from the medium-digestibility lines F2 and W64, and from the low-digestibility lines F271, L212, B73 and B14 is represented in FIG. 3.

The analyses carried out on 37 corn lines or ecotypes have allowed the inventors to show that 5 of the polymorphic sites between the F2 and F7 lines are in linkage disequilibrium with the insertion of the MITE transposon at the S0465 site (the naming of the polymorphic sites used here refers to their position with respect to the sequence alignment in FIG. 3; thus, the S0465 site corresponds to nucleotide 465 according to the numbering of this alignment). These sites are: the S0061 site, where a C in the sequence of F2 is replaced with a T in the sequence of F7; the S0231 site, where a T is inserted into the sequence of F7 with respect to the sequence of F2; the S0447 site, corresponding to a G in the sequence of F2 and to an A in the sequence of F7; the S0797 site, corresponding to a G in the sequence of F2 and to a T in the sequence of F7; and the S1208 site, corresponding to the deletion of 4 base pairs GCAT in the sequence of F7 with respect to the sequence of F2. Each of these polymorphisms characterizes a group of 5 related lines (F7, F7012, F324, F227 and F226) having a high degree of digestibility.

The inventors have also identified other polymorphic sites that appear to be associated with cell wall digestibility without being in linkage disequilibrium with the insertion of the MITE transposon at the S0465 site. They are: the S0270 site, corresponding to a deletion of one base (C), characteristic of a group of four high-digestibility lines (F564, EP1, Wis94-443 and Wis93-3520, not represented in FIG. 3), with respect to the sequence of F2; the S0988 site, where a G in the sequence of F2 is replaced with a C in the sequence of F7; this SNP affects a putative N-glycosylation site; and the S1663 site, located in the 3′ untranslated region and corresponding to an A in the sequence of F2, and a G in the sequence of F7, and which explains 20% of the phenotypic variability.

The demonstration by the inventors of the involvement of Pox3/U19 in the digestibility provides a set of means for obtaining plants, in particular monocotyledonous plants, especially corn, sorghum or panicum, having increased digestibility.

These novel means form the subject of the present invention.

A subject of the present invention is a method for improving the digestibility of a plant, wherein the expression and/or the activity of the Pox3/U19 peroxidase of said plant are totally or partially inhibited.

The term “Pox3/U19 peroxidase” is here defined as any protein having peroxidase activity and the polypeptide sequence of which exhibits at least 75%, preferably at least 85%, advantageously at least 94%, and entirely preferably at least 95%, identity with the sequence SEQ ID NO: 2 over as large a window of comparison as possible, preferably corresponding to the entire sequence SEQ ID NO: 2.

Unless otherwise specified, the percentage identities indicated here are established by means of the BLAST2 program (ALTSCHUL et al., Nucleic Acids Res., 25, 3389-3402, 1997) using the default parameters.

The total or partial inhibition of the expression and/or of the activity of the Pox3/U19 peroxidase can be obtained in various ways, by methods known in themselves.

Particularly advantageously, this inhibition can be obtained by intervening upstream of the production of the Pox3/U19 peroxidase, by mutagenesis of the gene encoding this protein, or else by inhibition or modification of the transcription or of the translation.

The mutagenesis of the gene encoding the Pox3/U19 peroxidase can take place at the level of the coding sequence or of the regulatory sequences for expression, in particular of the promoter. It is, for example, possible to delete all or part of said gene and/or to insert an exogenous sequence. By way of example, mention will be made of insertional mutagenesis: a large number of individuals derived from a plant that is active in terms of the transposition of a transposable element (AC or Mutator element in corn) are produced, and the plants in which there has been an insertion in the Pox3/U19 peroxidase gene are selected, for example by PCR.

It is also possible to introduce one or more point mutations with physical agents (for example radiations) or chemical agents. The consequence of these mutations is to shift the reading frame and/or to introduce a stop codon into the sequence and/or to modify the level of transcription and/or of translation of the gene and/or to make the enzyme less active than the wild-type protein. The mutated alleles of the Pox3/U19 gene can be identified, for example by PCR, using primers specific for said gene.

In this context, use may in particular be made of techniques of the “TILLING” type (Targeting Induced Local Lesions IN Genomes; McCALLUM et al., Plant Physiol., 123, 439-442, 2000).

Site-directed mutagenesis, targeting the gene encoding the Pox3/U19 peroxidase, can also be carried out. The inhibition or the modification of transcription and/or of translation can be obtained by the expression of sense, antisense or double-stranded RNA derived from the Pox3/U19 peroxidase gene, or of the cDNA of this protein, or else by the use of interfering RNAs (for review regarding antisense inhibition techniques, cf. for example: WATSON and GRIERSON, Transgenic Plants: Fundamentals and Applications (HIATT, A, ed) New York: Marcel DEKKER, 255-281, 1992; CHICAS and MACINO, EMBO reports, 21(11), 992-996, 2001; for review regarding more specifically the use of interfering RNAs, cf. HANNON, Nature, 418, 244-251, 2002).

A subject of the present invention is also the use of at least one polynucleotide chosen from:

a) a polynucleotide encoding a Pox3/U19 peroxidase as defined above;

b) a polynucleotide complementary to a polynucleotide a) above;

c) a fragment of at least 12 consecutive nucleotides, preferably at least 15, advantageously at least 20, and entirely preferably at least 50 consecutive nucleotides, specific for a polynucleotide a) or b) above, or capable of hybridizing selectively with said polynucleotide,

for obtaining a plant having increased digestibility.

The expression “polynucleotide encoding a Pox3/U19 peroxidase” is here defined as any poly-nucleotide containing the genetic information for the synthesis of said peroxidase.

This encompasses in particular the genomic DNA, for example the sequence SEQ ID NO: 1 represented in the appendix, and also the corresponding cDNA.

The expression “fragment specific for a polynucleotide a) or b) above” is defined as any fragment of said polynucleotide for which the sequence is not found in other genes of the same plant, and in particular in other genes of said plant encoding peroxidases.

The expression “polynucleotide capable of hybridizing selectively with a polynucleotide a) or b) above” is here defined as any polynucleotide which, when it is hybridized under stringent conditions with a library of nucleic acid from the same plant (in particular a genomic DNA or cDNA library), produces a detectable hybridization signal (i.e. at least twice as much as, preferably at least 5 times more than, the background noise) with said polynucleotide, but produces no detectable signal with other sequences of said library, and in particular with sequences encoding other peroxidases.

Stringent hybridization conditions, for a given polynucleotide, can be identified by those skilled in the art according to the size and the base composition of the polynucleotide concerned, and also according to the composition of the hybridization mixture (in particular pH and ionic strength). Generally, stringent conditions, for a polynucleotide of given size and given sequence, are obtained by carrying out the procedure at a temperature approximately 5° C. to 10° C. below the melting temperature (Tm) of the hybrid formed, in the same reaction mixture, by this polynucleotide and the sequence complementary thereto.

By way of example of a fragment specific for a polynucleotide a) or b) above, or capable of hybridizing selectively with said polynucleotide, mention will in particular be made of a polynucleotide that can be obtained from corn cDNA or nonintronic genomic DNA, by amplification under stringent conditions with the primers:

| | |
|---|---|
| OL 321: | |
| CACCGGAGTGGCTGCG | (SEQ ID NO: 5) |
| and | |
| OL 322: | |
| ATCGACAAATATATATGTTTATAAGG, | (SEQ ID NO: 6) |

and also the fragments of at least 12 consecutive nucleotides, preferably at least 15, advantageously at least 20, and entirely preferably at least 50 consecutive nucleotides, of said polynucleotide.

The positions of the primers SEQ ID NO: 5 and SEQ ID NO: 6 are boxed in on the sequence represented in FIG. 1.

A subject of the present invention is in particular a method for increasing the digestibility of a plant, by total or partial inhibition of the endogenous Pox3/U19 peroxidase of said plant, which method comprises the transformation of said plant with a recombinant DNA construct comprising a polynucleotide as defined above, placed in the sense orientation or in the antisense orientation, or that can be transcribed into double-stranded RNA, under the transcriptional control of a suitable promoter.

A subject of the present invention is also recombinant DNA constructs comprising a polynucleotide as defined above. These constructs may in particular be:

expression cassettes comprising a poly-nucleotide as defined above, under the transcriptional control of a suitable promoter. The expression cassettes can also advantageously comprise other regulatory elements, in particular regulatory elements for transcription such as terminators, enhancers, etc;

recombinant vectors comprising a poly-nucleotide, or advantageously an expression cassette, as defined above.

Recombinant DNA constructs in accordance with the invention can also comprise other elements, for example one or more selection markers.

Those skilled in the art have available to them a very wide choice of elements that can be used for obtaining recombinant DNA constructs in accordance with the invention.

By way of nonlimiting examples of promoters that can be used in the context of the present invention, mention will be made:

of constitutive promoters, such as the cauliflower mosaic virus (CaMV) 35S promoter described by KAY et al. (Science, 236, 4805, 1987), or its derivatives, the cassava vein mosaic virus (CsVMV) promoter described in PCT application WO 97/48819, the ubiquitin promoter or the rice “Actin-Intron-actin” promoter (McELROY et al., Mol. Gen. Genet., 231, 150-160, 1991; GenBank accession number S 44221);

inducible promoters or tissue-specific promoters, so as to modify the lignin content or composition only at certain developmental stages of the plant, under certain environmental conditions, or in certain target tissues, for instance stems, leaves, seeds, spathes, cortex or xylem.

By way of nonlimiting examples of other regulatory elements for transcription that can be used in the context of the present invention, mention will be made of terminators, such as the 3′NOS terminator of nopaline synthase (DEPICKER et al., J. Mol. Appl. Genet., 1, 561-573, 1982), or the CaMV 3′ terminator (FRANCK et al., Cell, 21, 285-294, 1980; GenBank accession number V00141).

By way of nonlimiting examples of selection marker genes that can be used in the context of the present invention, mention will in particular be made of genes that confer resistance to an antibiotic (HERRERA-ESTRELLA et al., EMBO J., 2, 987-995, 1983), such as hygromycin, kanamycin, bleomycin or streptomycin, or to a herbicide (EP 0 242 246), such as glufosinate, glyphosate or bromoxynil, or the NPTII gene which confers kanamycin resistance (BEVAN et al., Nucleic Acid Research, 11, 369-385, 1984).

The transformation of the plants can be carried out by many methods, known in themselves to those skilled in the art.

It is, for example, possible to transform plant cells, protoplasts or explants, and to regenerate a whole plant from the transformed material. The transformation can thus be carried out, by way of nonlimiting examples:

by transfer of the vectors in accordance with the invention into protoplasts, in particular after incubation of the latter in a solution of polyethylene glycol (PG) in the presence of divalent cations (Ca²⁺) according to the method described in the article by KRENS et al. (Nature, 296, 72-74, 1982);

by electroporation, in particular according to the method described in the article by FROMM et al. (Nature, 319, 791-793, 1986);

by using a gene gun that allows metal particles coated with the DNA sequences of interest to be projected at very high speed, thus delivering genes into the cell nucleus, in particular according to the technique described in the article by FINER et al. (Plant Cell Report, 11, 323-328, 1992);

by cytoplasmic or nuclear microinjection.

Agrobacterium tumefaciens can also be used, in particular according to the methods described in the articles by BEVAN et al. (Nucleic Acid Research, 11, 369-385, 1984) and by AN et al. (Plant Physiol., 81, 86-91, 1986) or else Agrobacterium rhizogenes can be used, in particular according to the method described in the article by JOUANIN et al. (Plant Sci., 53, 53-63, 1987). For example, the transformation of plant cells can be carried out by transfer of the T region of the Agrobacterium tumefaciens circular extrachromosomal tumor-inducing Ti plasmid using a binary system (WATSON et al., Ed. De Boeck University, 273-292, 1994). Agrobacterium tumefaciens can also be used on whole plants, for example by deposition, at the injury of a monocotyledonous plant, of the bacterium harboring the DNA to be transferred, in the presence of substances released at the injury of a dicotyledonous plant.

A subject of the present invention is also the plant cells and the transgenic plants that can be obtained by means of a method in accordance with the invention. Of course, the present invention encompasses the descendants, in particular the hybrids derived from a cross involving at least one plant according to the invention, obtained by sowing or by vegetative multiplication, of the plants directly obtained by the method of the invention. Preferably, said plants are monocotyledons, advantageously corn, sorghum or panicum plants.

The invention also comprises the plant cells and tissues, and also the organs or parts of plants, including leaves, stems, roots, flowers, fruits and/or seeds, obtained from a plant in accordance with the invention.

A subject of the present invention is also a method for selecting plants, which method comprises the search, in the plants to be tested, for an allele of the Pox3/U19 peroxidase gene having a mutation resulting in total or partial inhibition of the expression and/or of the activity of said protein.

Said allele can be sought by direct detection of the mutation responsible for the inhibition; it can also be sought by detection of the allelic form, associated with this mutation, of a polymorphism in linkage disequilibrium with it. For example, the insertion of the MITE transposon can be detected directly, or else by detection of the allelic form of one or more of the polymorphisms S0061, SS0231, S0447, S0797 and S1208.

The digestibility-favorable mutant alleles thus identified can then be introgressed into chosen lines, and in particular into “elite lines”, i.e. lines that have a substantial agronomic and commercial potential.

In the context of this method, use may in particular be made of polynucleotides specific for the Pox3/U19 peroxidase, as defined above, and especially:

primers for selectively amplifying the Pox3/U19 peroxidase gene or nucleic acid probes for selectively detecting this gene.

By way of nonlimiting examples of primers for selectively amplifying the Pox3/U19 peroxidase gene, mention will in particular be made of the pair of primers defined by the following sequences:

| | |
|---|---|
| GACGAAGCGGCACTGCTTGCGCTTCACCA | (SEQ ID NO: 7) |
| and | |
| TGCCACAGTAACAAGCGAGCTTACCAAGA. | (SEQ ID NO: 8) |

The positions of these primers are indicated in gray and underlined on the sequences represented in FIGS. 1 and 2.

The use of these primers makes it possible, by comparison of the amplification product with that obtained from plants having an active Pox3/U19 peroxidase, to detect the mutations that may affect the expression or the activity of said protein. For example, comparison of the sizes of the amplification products makes it possible to detect the presence of insertions or deletions capable of resulting in the production of an inactive protein;

nucleic acid primers or probes for detecting a given mutation, identified beforehand as affecting the expression or the activity of the Pox3/U19 peroxidase, or nucleic acid probes for selectively detecting this gene:

By way of nonlimiting example, mention will in particular be made of the pair of primers defined by the following sequences, which makes it possible to selectively amplify the DNA of plants having the insertion of the MITE transposon in the gene encoding Pox3/U19:

| | |
|---|---|
| GGCACTGGAGGCTCAGGGTGTGTT | (SEQ ID NO: 9) |
| AGGAGACAACGCCGGGGCAC. | (SEQ ID NO: 10) |

These two types of primers can be used separately or in combination; for example, the combination of a pair of primers for selectively amplifying the Pox3/U19 peroxidase gene and of a pair of primers for detecting a given mutation can be used for detecting plants that are heterozygous for this mutation.

A subject of the invention is also the pairs of primers defined above, and also the kits comprising these pairs of primers individually or in combination.

The present invention will be understood more clearly from the additional description which follows, which refers to nonlimiting examples illustrating the involvement of the Pox3/U19 peroxidase in digestibility, and its use for obtaining plants having improved digestibility.

### EXAMPLE 1

### Obtaining the POX3/U19 Genomic DNA

Primers specific for Pox3/U19 were defined from the cDNA sequence of the Pox3/U19 peroxidase.

The sense primer (U19S1) located at positions 1 to 29 of the cDNA is represented by the sequence (SEQ ID NO: 7) below:

5′-GACGAAGCGGCACTGCTTGCGCTTCACCA-3′
(29 bases Tm = 75° C.).

The antisense primer (U19AS1) located at positions 1170-1198 of the cDNA is represented by the sequence (SEQ ID NO: 8) below:

5′-TGCCACAGTAACAAGCGAGCTTACCAAGA-3′ (29 bases Tm=71° C.)

The position of these primers is indicated in FIG. 1.

The PCR amplifications are carried out using 100-150 ng of DNA, according to the following protocol:
PCR mix:
100-150 ng of genomic DNA
0.2 μM of each primer
200 μM of each DNTP
2.5 units of REDTaq® polymerase (Sigma)/50 μl of reaction 5 μl of 10× buffer, pH 8.3, comprising 100 mM of tris-HCL, 500 mM of KCl, 15 mM of MgCl₂ and 0.01% of gelatin
Cycle:
5 min at 95° C.
30 cycles: 30 sec at 95° C.
   30 sec at 60° C.
   1 min 40 at 72° C.
   5 min at 72° C.

The sequence obtained by amplification is approximately 1.44 kb in size. It is made up of 3 exons and 2 introns, respectively of approximately 130 and 100 bp. The monocotyledon consensus splice sites are present.

### EXAMPLE 2

### Demonstration of the Association of an Improvement in Digestibility with an Inactive POX3/U19 Peroxidase

Polymorphic Sites/Digestibility Association

The sequence encoding the Pox3/U19 peroxidase was amplified in 37 lines. The sequences thus obtained were aligned. The percentage polymorphism is 2.2%, i.e. 1 SNP approximately every 45 bases. The degree of polymorphism of the amino acid sequence is 1.39%, i.e. only 5 amino acid changes out of 358.

The construction of a phylogenetic tree according to the UPGMA method on the nucleic acid sequences makes it possible to distinguish two groups:

The first group comprises the F7 line, and related lines for which the PCR amplification of the gene encoding Pox3/U19 results in a fragment of 1744 bp.

The second group comprises the lines for which the PCR amplification of the gene encoding Pox3/U19 gives a fragment of approximately 1410 bp.

This difference in size between the amplified products of the two groups is due to the presence, in the second exon, of a 321 bp element having the structural characteristics of a transposable element. In fact, at each of its ends, about fifteen base pairs are repeated in an imperfect and inverted manner. A direct repeat of 5 base pairs is present on either side of the element insertion site. This corresponds to an MITE element (Miniature Inverted-repeat Transposable Element) (WESSLER et al., Current Opinion in Genetics and Development, 5, 814-821, 1995).

An analysis of variance (ANOVA test) carried out on each polymorphic locus makes it possible to investigate associations with the digestibility parameter. Next, a step consisting of regression by the least squares method (for a linear model) is carried out in order to pinpoint the locus that explains the majority of the variability of the digestible nature.

The result at the locus for insertion of the MITE transposon is as follows: the probability of the polymorphism corresponding to the insertion of the element being linked to the digestibility is significant with a threshold of 5% (probability of 0.032).

Investigation of Association Between Digestibility and the Presence of the MITE Insertion

A pair of primers that specifically amplifies the MITE element insertion was defined.

The sense primer is represented by the sequence (SEQ ID NO: 9) below:

U19MITES
5′-GGCACTGGAGGCTCAGGGTGTGTT-3′
(24 bases, Tm = 67.8° C.),

and the antisense primer is represented by the sequence (SEQ ID NO: 10) below:

U19MITEAS
5′-AGGAGACAACGCCGGGGCAC-3′
(20 bases, Tm = 65.5° C.)

The PCR amplifications are carried out using 100 ng of DNA, according to the following protocol:
PCR mix:
100 ng of genomic DNA
0.2 μM of each primer
200 μM of each dNTP
1.25 units of REDTaq® polymerase (Sigma)/25 μl of reaction
2.5 μl of 10× buffer, pH 8.3, comprising 100 mM of tris-HCL, 500 mM of KCl, 11 mM of MgCl₂ and 0.01% of gelatin
Cycle:
5 min at 95° C.
25 cycles: 30 sec at 95° C.
   30 sec at 65° C.
   30 sec at 72° C.
   5 min at 72° C.

This pair of primers specifically amplifies the MITE insertion in the Pox3/U19 gene. There is no amplification with this pair of primers when the individuals do not have this mutation in the Pox3/U19 gene.

Characteristics of the Peroxidase of the F7 Line

The F7 line and certain related lines, for which the gene encoding the peroxidase has been sequenced, have a sequence of 1744 bp instead of 1440 bp. The genomic sequence obtained is made up of the coding region consisting of three exons, and of two introns. The introns are small in size, i.e. 130 and 100 bp respectively.

The difference is size is due to the insertion into the second exon of a 321 bp transposon of MITE type.

Translation of the Pox3/U19 allele possessing the MITE element insertion makes it possible to obtain an amino acid sequence homologous to the translations of the Pox3/U19 alleles that do not have this insertion for the first 111 amino acids, corresponding to the translation of the first exon and of the first third of the second exon. In fact, the insertion of the MITE element introduces a stop codon into the sequence 75 nucleotides after the beginning of the insertion.

It therefore appears that, unlike the wild-type peroxidase that contains 358 amino acids, the peroxidase of the F7 line and of the lines from which the gene is interrupted by the insertion of the MITE contains only 111+25 amino acids. The insertion of the transposable element bring about, in the translation product, the deletion of amino acids putatively involved in the catalytic activity. This is because, by bioinformatic analysis (Prosite release 10.0), the peroxidase domain 1 is thought to be located from positions 163 to 212, and the peroxidase domain 2 from positions 36 to 87. Consequently, insertion of the MITE element would prevent translation of the peroxidase domain 1 and would render the enzyme totally or partially inactive.

Genotyping of Corn Lines

The genotyping is carried out by PCR on a few lines (F7012, Lan496 and F192) and then on a wider collection.

The primers used are U19S, U19AS, U19MITES and U19MITEAS. According to the pair of primers used, it is possible to have a dominant or codominant marker. For example, the U19S/U19MITEAS pair makes it possible to distinguish the plants that are homozygous for the mutated or wild-type allele from the heterozygous plants.

F7012 is a fixed (homozygous) line derived from F7 which possesses the MITE element. Lan496 is also a fixed line not related to F7, and which does not possess the insertion. The hybrid exhibits the 2 alleles of the Pox3/U19 gene. F192, which is a fixed line derived from the F7×F2 line, has been typed and exhibits the insertion of the MITE element.

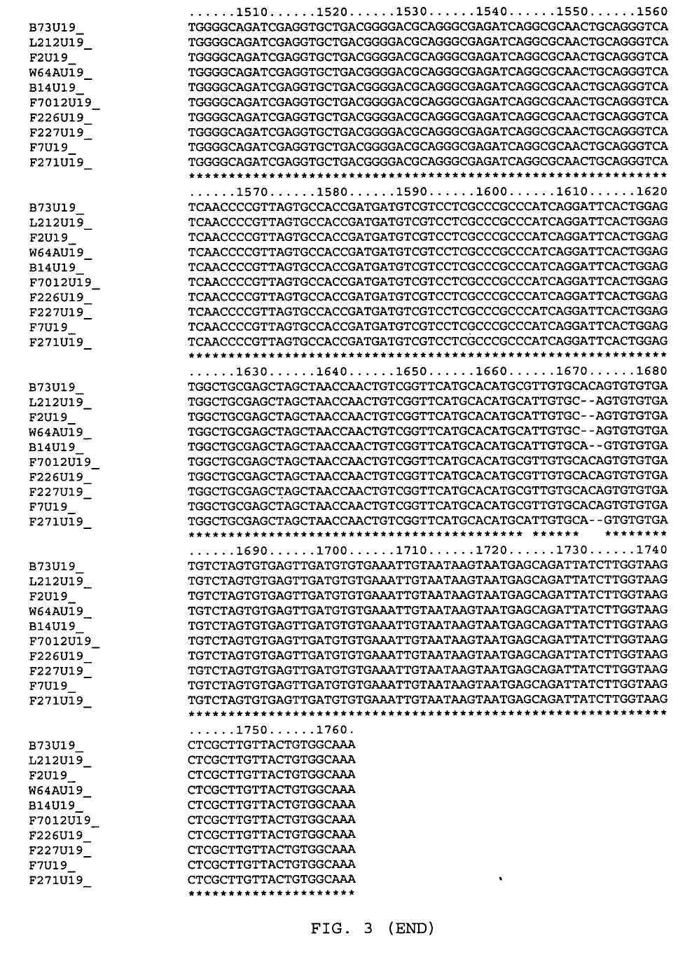

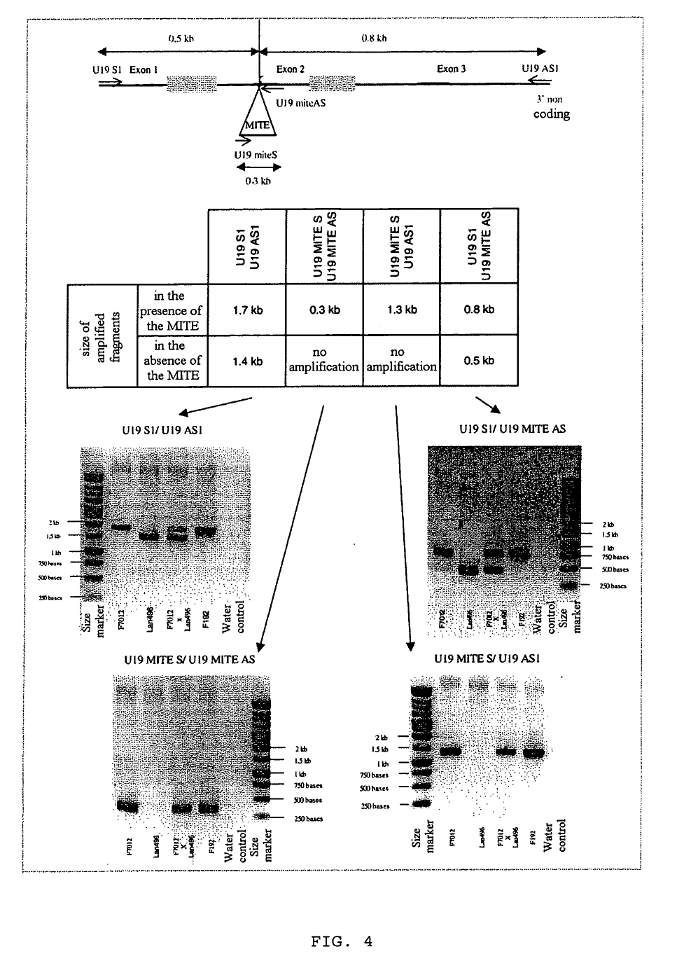

The results expected for each pair of primers are summarized in FIG. 4.

In a second step, a wider collection of lines having the F7 parent in their genealogy and having a variable level of digestibility was typed.

The results of the typing for the individuals related to F7 and the corresponding digestibility marks are represented in table I hereinafter.

The lines were marked:

1 when the MITE element is present in the Pox3/U19 gene;

0 when the MITE element is absent from the Pox3/U19 gene.

TABLE I

| | MITE | |
|---|---|---|
| | presence/absence | Digestibility |
| F7 | 1 | 4 |
| F192 | 1 | 3 |
| F7012 | 1 | 4.5 |
| F226 | 1 | 3.5 |
| F227 | 1 | 3 |
| F324 | 1 | 5.5 |
| 2058 | 1 | 4.5 |
| 2068 | 1 | 5 |
| LGFS | 1 | 3.5 |
| CP1718 | 0 | 3 |
| CP1622 | 0 | 3 |
| SK02 | 0 | 3.5 |
| SK122 | 0 | 2.5 |
| SK132 | 0 | 3 |
| F268 | 0 | 2.5 |
| F7023 | 0 | 1.5 |
| LGD3 | 0 | 2.5 |
| LGI9 | 0 | 1.5 |
| LGI2 | 0 | 2 |
| LGI1 | 0 | 3 |

The digestibility mark comes from the long-term experiments carried out since 1992. The lines were phenotyped in terms of individual value for their in vitro wall digestibility value (DINAG criterion, ARGILLIER et al., Euphytica, 82, 175-184, 1995). These values were then standardized and summarized in the form of a mark of from 1 (barely digestible, such as F271) to 5 (very digestible, such as F324).

Table II hereinafter illustrates the comparison of the means of the digestibility marks for the individuals having the MITE insertion and for the individuals that do not have it.

TABLE II

| | Digestibility mean | Standard deviation | Variance | Number of lines |
|---|---|---|---|---|
| Lines | 4.06 | 0.88 | 0.78 | 9 |
| possessing | | | | |
| the MITE | | | | |
| Lines not | 2.55 | 0.65 | 0.42 | 11 |
| possessing | | | | |
| the MITE | | | | |

Comparison of means

| | | | F | | F | |
|---|---|---|---|---|---|---|
| | | | tabulated | | tabulated | |
| Estimation | | Degrees | for a | | for a | |
| of common | | of | threshold | | threshold | |
| variance | F calculated | freedom | of 5% | Significant ? | of 1% | Significant ? |
| 0.58 | 4.41 | 18 | 2.101 | YES | 2.878 | YES |

The test for comparison of means between the lines that possess the MITE insertion and those that do not possess it shows that the means of the digestibility marks for the lines that have and that do not have the MITE element are significantly different at a threshold of 1%.

The analysis of variance carried out with, as covariable, the percentage of F7 in the lines analyzed, confirms a highly significant effect of the MITE insertion.

These results show an association between the presence of an inactive peroxidase and an increased level of digestibility.

Association Between Digestibility and the Presence of the MITE Insertion in a Lan496×F7012 Recombinant Population

The impact of the mutated Pox3/U19 allele was evaluated in another way by typing a population of doubled haploids derived from the crossing of Lan496 (absence of insertion of the MITE element and with medium digestibility) with F7012 (insertion of the MITE element and with good digestibility):

46 doubled haploids were typed 0,

48 doubled haploids typed 1.

The segregation is of ½ ½ type.

The effect of the insertion of the MITE element on the wall characteristics (digestibility, lignification, parietal carbohydrate composition) was studied based on the estimation of these characteristics made on plants harvested at a normal ensilage date in 2002 (September 10, 2002). This being so, the difference in earliness of flowering between the parents and the conditions relatively unfavorable to corn maturation in 2002 meant that, at harvest, there was a considerable difference in level of maturation between the various lines studied.

The parietal characteristics and the digestibility values were evaluated by NIRS (Near Infra Red Spectroscopy), using the calibration of the Centre de Recherches Agronomiques [Agronomic Research Center] in Libramont, Belgium.

The NDF, ADF and ADL measurements were carried out according to the protocols described in GOERING et al., Agric. Handb., 379, US Gov. Print Office, Washington D.C., 1971; those of Klason lignin according to DENCE et al., Methods in Lignin Chemistry Springer (ed.) Berlin, 33-62, 1992; the DINAG and DINAGZ measurements, respectively, according to ARGILLIER et al., Euphytica, 82, 175-184, 1995 and BARRIERE et al., Fourrages, 163, 221-238, 2000. Finally, the dNDF measurements are carried out according to STRUIK, doctoral thesis, University of Wageningen, 1983 and DOLSTRA and MEDEMA, Proceedings of The 15th Congress Maize And Sorghum Section of Eucarpia, Jun. 4-8, 1990, Baden, Austria, 258-270.

An analysis of variance was carried out with block, subblock, solids, MITE marker and genotype effects, a solids covariable being necessary due to the shift in earliness between the two parents, in particular after a cold summer relatively unfavorable to the maturation of the lines. The results obtained with the “MITE marker” variable are given in the table below.

TABLE III

| | | Residual mean | | |
|---|---|---|---|---|
| MITE marker | MITE mean square | square | F (Fisher) | P (probability in %) |
| ADL/NDF | 0.28 | 0.14 | 2.0 | 16.0 ns |
| LK/NDF | 30.5 | 0.65 | 47.1 | 0.00** |
| NDF | 92.9 | 2.23 | 41.5 | 0.00** |
| ADF | 52.0 | 1.15 | 45.2 | 0.00** |
| NDF-ADF | 38.4 | 0.87 | 43.9 | 0.00** |
| ADF-ADL | 5.9 | 0.35 | 17.0 | 0.00** |
| DINAGZ | 89.7 | 1.33 | 67.4 | 0.00** |
| dNDF | 26.6 | 2.93 | 9.1 | 0.39** |

NDF: wall content
ADF: cellulose and ADL lignin content
NDF-ADF: hemicellulose content
ADL/NDF: ADL lignin content
LK/NDF: Klason lignin content
dNDF: wall digestibility
DINAGZ: wall digestibility (except starch, soluble carbohydrate)
ns: non significant (P > 10%)
**significant at the threshold of 1%

At a harvesting stage representative of the ensilage stage (between 30 and 35% of solids on average), the effect of the MITE measured by the criterion of the Fisher F test is very significant on many characteristics linked to wall digestibility. There is thus a significant effect of the MITE insertion on the DINAGZ and dNDF in vitro wall digestibility characteristics. Similarly, there is an effect of the MITE on the composition of parietal carbohydrates hemicellulose and cellulose. On wall lignification, the effect of the MITE is very significant on total lignin, estimated by the Klason lignin criterion in the NDF, but is not significant on the most resistant part of the lignin estimated by the ADL in the NDF.

Presence of the Pox3/U19 Peroxidase RNA in F7012 (Possessing the MITE Insertion) and in Lan496, by RT-PCR

In order to confirm the inactivation of the peroxidase by the insertion of an MITE element, RT-PCR analyses were carried out on the top and the bottom of young plants (mixture of stems and leaf sheaths) and on leaf blades for plants at the same stage. These analyses were carried out on the F7012 line (carrying the MITE insertion) and on the Lan496 line. Total RNA was extracted from the tissues in the presence of stainless steel beads in 2 ml Eppendorf tubes soaked in liquid nitrogen. The tissues were then ground in an MM300 mixer mill (Qiagen®), agitating for twice 30 seconds. The powder thus obtained is vortexed with 1 ml of TRIzol® reagent (Invitrogen) at ambient temperature. The mixture is centrifuged at 18 000 g for 10 minutes at 4° C. The aqueous phase is again extracted at ambient temperature with 200 μl of chloroform. The RNA is then precipitated with 500 μl of isopropanol for 10 minutes at ambient temperature. After centrifugation for 10 minutes (18 000 g, 4° C.), the RNA pellet is washed with 1 ml of 70% ethanol, dried, and then suspended in 30 μl of RNAse-free water. After treatment with DNAse subsequently inactivated in accordance with the supplier's (AMBION) instructions, the RNA is quantified in a spectro-photometer at 260 nm. Approximately 5 μg of total RNA are reverse transcribed using random hexamers (Amersham) and a reverse transcriptase without RNaseH activity (Fermentas). The 20 μl of the reverse transcription reaction also contain 2.5×10⁵ copies of GeneAmplimer pAW109 RNA (Applied Biosystems). The cDNA thus obtained is diluted 50 times in water. 5 μl are used for the PCR reaction in a total volume of 20 μl.

The Pox3/U19 allele is amplified using the U19S1 primer (SEQ ID NO: 7) with: either the U19R1 primer (SEQ ID NO: 11: 5′-CGTCAGGTTGCCTACCGTGTCGATCAGCAC-3′) located 84 bp upstream of the MITE insertion, or the U19MITEAS primer (SEQ ID NO: 10) located downstream of the insertion. The amplification is carried out with 18S rRNA and GeneAmplimer pAW109 RNA as positive control. The number of cycles is adjusted according to the visualization of visible bands on agarose gel, in order to have a semi-quantitative evaluation. The bands are visualized with ethidium bromide.

No difference in signal intensity is visible between F7012 and Lan496 for the portion upstream of Pox3/U19, whereas the bands derived from the amplification with the primers surrounding the MITE are barely visible. This difference in intensity can be explained by a rapid degradation of the untranslated portion of the RNA of the mutant allele. This experiment confirms that the insertion of the MITE transposon results in the inactivation of the Pox3/U19 peroxidase.

### EXAMPLE 3

### Improvement in Digestibility by Inactivation of the POX3/U19 Peroxidase

For this approach, a bioanalytical study was carried out beforehand in order to search for a region specific for the U19 peroxidase so as to deregulate only a single gene of this multigene family. After cloning of this fragment, it was verified, by Southern blotting, that this fragment hybridized only a single locus.

Agrobacterium tumefaciens Transformation of Corn Plants with a Pox3/U19 Gene Antisense

Construction of a Plasmid Comprising the 3′UTR Sequence of the Pox3/U19 Peroxidase in the Antisense Orientation:

The vector used for transforming the corn with Agrobacterium tumefaciens is in the form of a superbinary plasmid of approximately 50 kb (pREC 520).

This vector contains:
an ori region: Col EI plasmid origin of replication, necessary for maintenance and multiplication of the plasmid in Escherichia coli. This origin of replication is not functional in Agrobacterium tumefaciens,
an origin of replication that is functional in Agrobacterium tumefaciens and in Escherichia coli,
the cos region of the lambda bacteriophage, that may be useful for manipulating the vector in vitro,
the additional virB, virC and virG regions of Agrobacterium tumefaciens which increase the transformation efficiency,

the gene for resistance to tetracycline (Tetra) and to spectinomycin (Spect) which are only expressed in bacteria,

a T-DNA carrying two expression cassettes: one contains the CsVMV promoter, the 3′UTR sequence of Pox3/U19 in the antisense orientation and the NOS terminator, and the other contains a selection gene (for example, a gene for resistance to herbicides) under the control of the rice actin promoter and followed by the 3′NOS terminator.

Protocol for Transformation with Agrobacterium tumefaciens

(according to ISHIDA et al., Nature Biotechnology, 14, 745-750, 1996).

Immature ears from a line produced under glass are removed 10 days after pollination and sterilized for 15 minutes. The embryos are removed and brought into contact for 5 minutes with a suspension of Agrobacterium containing the superbinary vector as described above. After having been removed from the suspension of Agrobacterium, the embryos are placed in culture on a medium containing neither bacteriostatic nor selective agent. This coculture takes place in the dark for 4 to 7 days.

After the coculture, the embryos are subcultured on a fresh callogenesis medium containing the bacteriostatic and the selective agent. A callus will be initiated and develop from transformed cells of these embryos. The callogenesis step takes place at 25° C. in the dark and lasts 5 weeks. The embryo-calluses are subcultured on fresh medium every 2 to 3 weeks.

At the end of this step, the transformed white calluses are excised from the primary explant and are subcultured on a regeneration medium containing zeatin instead of auxin. The regeneration step also lasts weeks, interspersed with subculturing of the callus on fresh medium every 2 to 3 weeks.

After 2-3 weeks on this medium, plantlets regenerate from the calluses. Once the plantlets are developed enough, they are rooted in tubes.

After 10-15 days in tubes, the plantlets are acclimatized in a phytotron before being transferred to a glasshouse. The transformants are then cultivated and crossed with pollen from a nontransgenic plant so as to produce the T1 generation.

Transformation of Corn Plants with a Pox3/U19 Gene Antisense, by Biolistics

Construction of a Plasmid Comprising the 3′UTR Sequence of the Pox3/U19 Peroxidase in the Antisense Orientation:

The 3′UTR region of Pox3/U19 in the antisense orientation was cloned into the vector pTriplEX2 (SMART™ cDNA library construction kit, CLONTECH). The Hind III—EcoR I fragment framing the sequence of interest was introduced into the vector E 919, also opened at the Hind III and EcoR I restriction sites, which sites are located, respectively, downstream of the CsVMV promoter and upstream of the NOS terminator. The plasmid of interest E 1105 is thus obtained.

The technique for transformation by biolistics involves co-transforming plant cells, firstly, with the gene of interest (E 1105) and, secondly, with a plasmid carrying an expression cassette (pDM302) comprising a selection gene (for example, a gene for resistance to a herbicide) preceded by the appropriate promoter and followed by the appropriate terminator.

Transformation Protocol

Immature HiII embryos are removed 10 days after pollination. They are placed in culture on an osmotic medium. 4 days later, they are bombarded with gold particles coated with plasmid containing the gene of interest and a plasmid carrying the selection gene.

The embryos are then subcultured on a callogenesis medium. This step, carried out in the dark and at 25° C., lasts approximately 2 months, interspersed with subculturing on fresh medium every 15 days. Once the transformed calluses are selected, they are cultivated on maturation medium, and then on regeneration medium. The regeneration step is carried out in the light and lasts 2 to 4 weeks.

As soon as 1 to 2 weeks after switching onto this medium, plantlets regenerate from somatic embryos initiated during the maturation step. These plantlets are then rooted in tubes. As in the case of the plants produced by transformation with Agrobacterium, the rooted plantlets are then acclimatized in a phytotron before being transferred into a glasshouse for production of T1 seeds.

Inactivation of the Pox3/U19 Peroxidase with iRNA

Construction of a Vector Comprising the 3′UTR Sequence of the Pox3/U19 Peroxidase in the Sense and Antisense Orientation

This vector is constructed using the Gateway® system (Invitrogen).

A 3′UTR fragment of the Pox3/U19 gene is amplified by PCR from corn cDNA contained in a plasmid called E1100.

The primers used are as follows:
Ol 321: (CACCGGAGTGGCTGCG; SEQ ID NO: 5) containing a CACC extension in the 5′ position, required for the cloning into the entry vector, and

Ol 322: (ATCGACAAATATATATGTTTATAAGG; SEQ ID NO: 6). The amplification conditions used are as follows:

| | | | |
|---|---|---|---|
| plasmid E 1100 (10 ng/μl): | 2 | μl | |
| 10x buffer (cloned Pfu buffer) | 2 | μl | |
| dNTP (5 mM each) | 0.8 | μl | |
| Ol 321 10 μM | 1 | μl | |
| Ol 322 10 μM | 1 | μl | |
| Pfu (2.5 U/μl)(Stratagene) | 1 | μl | |
| H₂O | 10.7 | μl | |
| Cycle: | 10 | min 95° | 20 times |
| | 30 | sec 92° | |
| | 30 | sec 55° | |
| | 40 | sec 72° | |
| | 10 | min 72°. | |

The amplified fragment is then cloned in the antisense and in the sense direction into the vector pENTR D/Topo (Invitrogen), so as to give the entry vector E1121.

In parallel, the destination vector E1122, which contains the rice tubulin intron, is constructed. A double recombination between these 2 vectors results in the vector E1129 being obtained, which vector contains a cassette comprising the 3′UTR of Pox3/U19 in the antisense orientation, the rice tubulin intron, and the 3′UTR of Pox3/U19 in the sense orientation. On either side of this cassette are two Sac I restriction sites. The Sac I fragment is cloned into an intermediate cloning vector carrying a kanamycin resistance gene. The vector obtained is called E 1137. The Sac I fragment of E 1137 is introduced into the vector E 919 open at the Sac I site, so as to obtain the vector E 1142. This vector carries an expression cassette consisting of the CsVMV promoter, of the 3′UTR sequence of U19 in the antisense orientation, of the first intron of the rice tubulin gene, of the 3′UTR sequence of Pox3/U19 in the sense orientation and of the NOS terminator.

It can be used for the transformation of corn by biolistics, as described above.

Obtaining the Plants

115 transgenic lines were obtained by following the deregulation protocol described above. Among them, 105 are undergoing observation in a glasshouse and 18 in the open field. These plants are the subject of phenotypic observations in addition to cross analyses consisting of histochemistry, of RT-PCR and of near infrared evaluation (NIRS). Subsequent to these various analyses, the lines selected are the subject of in vitro digestibility analyses (according to the various protocols mentioned in example 2 above).

What is claimed is:

1) A method for improving the digestibility of a plant, wherein the expression and/or the activity, in said plant, of a peroxidase, hereinafter referred to as Pox3/U19 peroxidase, the polypeptide sequence of which exhibits at least 75% identity with the sequence SEQ ID NO: 2, is totally or partially inhibited.

2) The method as claimed in claim 1, wherein said plant is corn.

3) The use of at least one polynucleotide chosen from:
a) a polynucleotide encoding a Pox3/U19 peroxidase as defined in claim 1;
b) a polynucleotide complementary to a polynucleotide a) above;
c) a fragment of at least 12 consecutive nucleotides, of a polynucleotide a) or b) above, or capable of hybridizing selectively with said polynucleotide,
for carrying out a method as claimed in either one of claims 1 and 2.

4) The use as claimed in claim 3, wherein said polynucleotide is chosen from:
a polynucleotide that can be obtained from corn cDNA or genomic DNA, by amplification with the primers

| | |
|---|---|
| CACCGGAGTGGCTGCG | (SEQ ID NO: 5) |
| and | |
| ATCGACAAATATATATGTTTATAAGG; | (SEQ ID NO: 6) |

a fragment of at least 12 consecutive nucleotides of said nucleotide.

5) The method as claimed in either one of claims 1 and 2, wherein the inhibition of the expression and/or of the activity of the Pox3/U19 peroxidase is obtained by mutagenesis of the gene encoding said peroxidase.

6) The method as claimed in either one of claims 1 and 2, which method comprises the transformation of said plant with a recombinant DNA construct comprising a polynucleotide as defined in either one of claims 3 and 4, under the transcriptional control of a suitable promoter.

7) An expression cassette comprising a polynucleotide as defined in either one of claims 3 and 4, under the transcriptional control of a suitable promoter.

8) A recombinant vector containing an expression cassette as claimed in claim 7.

9) A genetically modified plant that can be obtained by means of a method as claimed in claim 6.

10) A method for selecting plants, which method comprises the search, in the plants to be tested, for an allele of the Pox3/U19 peroxidase gene having a mutation resulting in total or partial inhibition of the expression and/or of the activity of said peroxidase.

11) The method as claimed in claim 10, which method is carried out on corn.

12) The use of at least one polynucleotide as defined in either one of claims 3 and 4, for carrying out a method as claimed in either of claims 10 and 11.

13) The use as claimed in claim 12, which use comprises the employment of the pair of primers SEQ ID NO: 7 and SEQ ID NO: 8.

14) The use as claimed in either one of claims 12 and 13, which use comprises the employment of the pair of primers SEQ ID NO: 9 and SEQ ID NO: 10.

15) A pair of primers of sequences SEQ ID NO: 5 and SEQ ID NO: 6.

16) A pair of primers of sequences SEQ ID NO: 7 and SEQ ID NO: 8.

17) A pair of primers of sequences SEQ ID NO: 9 and SEQ ID NO: 10.

18) A kit for carrying out a method as claimed in either one of claims 10 and 11, which kit comprises at least one pair of primers as claimed in either one of claims 16 and 17.
